(19) United States
(12) Patent Application Publication
(10) Pub. No.: US 2002/0015938 A1
(43) Pub. Date: Feb. 7, 2002

(54) HIGH-THROUGHPUT PREFORMULATION OF POTENTIAL DRUG CANDIDATES

(76) Inventor: NARMADA SHENOY, SUNNYVALE, CA (US)

Correspondence Address: BETH A. BURROUS OR JOHN P. ISACSON, FOLEY & LARDNER, WASHINGTON HARBOUR, 3000 K STREET, N.W. SUITE 500, WASHINGTON, DC, 20007-5109, US

(21) Appl. No.: 09/182,700

(22) Filed: Oct. 29, 1998

Related U.S. Application Data
(60) Provisional application No. 60/063,951, filed on Oct. 31, 1997.

(51) Int. Cl. C12Q 1/00; G01N 33/53; C07D 413/02; C 07D 4 1/02; A61K 31/404

(52) U.S. Cl. 435/4; 435/7.1; 544/143; 544/144; 546/201; 548/435; 548/460; 548/509; 548/511; 548/512; 548/518; 514/414; 514/422; 514/444; 514/445; 514/323; 514/469

(57) ABSTRACT

The invention relates to a method of simultaneous high-throughput preformulation quantification of potential drug candidates, where an aliquot of a mixture of solutions containing different compounds is injected into a high pressure liquid chromatograph. The concentration of each compound can be determined by high pressure liquid chromatographic analysis, and correlated to a physico-chemical property of the compound.

### RELATED APPLICATIONS

This application claims priority to the U.S. Provisional Application, Ser. No. 60/063,951, filed Oct. 31, 1997, by Shenoy et al., and entitled “HIGH-THROUGHPUT PREFORMULATION OF POTENTIAL DRUG CANDIDATES” (Lyon & Lyon Docket No. 230/106), which is hereby incorporated herein by reference in its entirety, including any drawings.

### FIELD OF THE INVENTION

The present invention features a method for high-throughput pharmaceutical preformulations.

### BACKGROUND OF THE INVENTION

The following description of the background of the invention is provided to aid in understanding the invention, but is not admitted to describe or constitute prior art to the invention.

In order to determine an appropriate formulation for a potential pharmaceutical, physico-chemical properties of a compound conventionally are analyzed in preformulation studies. One of the challenges of preformulation studies involves investigation of the physico-chemical properties, such as solubility, pKₐ, partitioning, pH-solubility, pH-stability, etc., of a large set of potential drug candidate in the shortest time and with optimum effort to value balance. Methods currently used in the art require that each compound be tested individually for a particular property, using high pressure liquid chromatography (HPLC). This process can be very time consuming if one is attempting preformulation studies on a large number of compounds. Thus, the need exists in the art for a high-throughput method that allows one to effectively and accurately study a group of compounds.

### SUMMARY OF THE INVENTION

The present invention is directed towards a method which allows for simultaneous preformulation study of a number of drug candidates. The methods of the present invention allow for rapid, efficient, and accurate determination of the physico-chemical properties of a large set of potential drugs in preformulation studies.

The term “high-throughput” in the context of this invention refers to the ability to test many compounds in a short period of time, preferably, simultaneously.

The term “preformulation” refers to a physical or chemical property of a compound that is used to determine its formulation as a drug. Thus, “preformulation study” refers to the determination of a physical or chemical property of a compound. “Formulation” refers to design of the final dosage form of a particular drug.

Thus, in a first aspect the invention provides a method of simultaneous evaluation of a plurality of potential drug candidates, comprising the steps of (a) forming multiple series of solutions, each series of solutions containing a different known compound in a solvent or in a mixture of solvents under varying solution conditions; (b) mixing a single solution from one series with the corresponding solution from each of the other series of solutions, thereby forming a mixture of solutions, where the corresponding solutions have substantially similar solution conditions; (c) separately injecting an aliquot of each of the mixture of solutions into a high pressure liquid chromatograph; (d) determining the concentration of each of the different compounds in each of the mixture of solutions by analysis of results from the injection; and (e) correlating the concentration of each of the different compounds in each of the mixture of solutions to a physico-chemical property of the compound.

The solution conditions of step (a) of the above method are preferably selected from the group consisting of pH, polarity, dielectric constant, and temperature.

The term “potential drug candidate” refers to a compound which potentially can be used as a drug against a disease. The pharmacological activities of the compound may be unknown.

In preferred embodiments, multiple series of solutions are prepared by dissolving a single known pure compound in each series of solutions (FIGS. 1a and 1b). Thus, each series of solutions contains a dissolved compound which is different than the compound dissolved in the other series of solutions. The solvent of each series of solutions may be an aqueous solvent, an organic solvent, or a mixture of both. Within each series of solvents one of the conditions of the solvent is varied. Thus, for example, within one series the pH of the solution is varied.

A single solution from one series is then mixed with the corresponding solution from each of the other series of solutions thereby forming a mixture of solutions. The corresponding solutions forming the mixture have substantially similar solution conditions (FIG. 1c).

By “substantially similar solution conditions” it is meant that the solution condition of the individual solutions forming the mixture is nearly identical. Thus, for example, the solutions forming the mixture will have nearly the same pH, nearly the same polarity, or nearly the same dielectric constant. By “substantially similar”, “nearly identical”, and “nearly the same” it is meant that the values determining the solution condition for the different solutions are within 5% of each other.

The term “series” with respect to solutions in the context of this invention refers to one or more flasks of a solution where a condition of the solution is varied within the series. Thus, for example, a number of flasks containing an aqueous solution with different pH values constitutes a series of solutions. Also, a mixture of organic and aqueous solvents within a flask constitutes a series of solutions in the context of this invention.

The term “compound” refers to the compound or a pharmaceutically acceptable salt, ester, amide, prodrug, isomer, or metabolite, thereof.

The term “pharmaceutically acceptable salt” refers to a formulation of a compound that does not abrogate the biological activity and properties of the compound. Pharmaceutical salts can be obtained by reacting a compound of the invention with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “prodrug” refers to an agent that is converted into the parent drug in vivo. Prodrugs may be easier to administer than the parent drug in some situations. For example, the prodrug may be bioavailable by oral administration but the parent is not, or the prodrug may improve solubility to allow for intravenous administration.

The term “solvent” refers to a liquid which has the ability to dissolve a compound to some extent. A “mixture of solvents” refers to a combination of two or more solvents which may or may not be miscible. By “miscible” it is meant that the mixture of two liquids form a homogeneous, monophasic combination.

The term “aliquot” means a small portion of a solution which has the same properties, both physical and chemical, as the entire solution. Thus, the concentration of a solute in an aliquot of the solution is the same as the compound's concentration in the entire solution.

The term “physico-chemical property” refers to the physical and chemical properties of a compound. These properties include, but are not limited to, the pKₐ, the solubility, and the partition coefficient.

The term “pKₐ” refers to the negative of the logarithm of the acidity constant of a compound. The term “acidity constant” refers to the equilibrium constant of a reaction of the compound in which the compound loses a proton to the solvent.

The term “pH” refers to the negative of the logarithm of the hydronium ion concentration in an aqueous solution.

The term “polarity” refers to a condition of a molecule where the center of negative charge in the molecule does not coincide with the center of positive charge in the molecule. The further away the two centers are, the more polar a molecule is.

The term “dielectric constant” refers to the ability of a solvent to lower the attraction of the oppositely charged particles within it. Higher dielectric constants correspond to lower attraction of the oppositely charged particles within the solution.

Alternatively, the invention provides for a method of simultaneous evaluation of a plurality of potential drug candidates, comprising the steps of (a) forming a mixture of different known compounds in a solvent or in a mixture of solvents under varying solution conditions to form a series of solutions; (b) separately injecting an aliquot of each solution in the series into an HPLC; (c) determining the concentration of each of the different compounds in each of the solutions in the series by analysis of the results from the injecting; and (d) correlating the concentration of each of the different compounds in each of the solutions in the series to a physico-chemical property of the compound.

In a preferred embodiment, the invention provides an indolinone compound of formula I

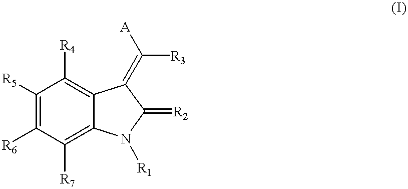

where

a) R₁ and R₃ are independently selected from the group consisting of hydrogen and saturated or unsaturated alkyl;

b) R₂ is selected from the group consisting of oxygen and sulfur;

c) R₄, R₅, R₆, and R₇ are independently selected from the group consisting of

(i) hydrogen;

(ii) saturated or unsaturated alkyl;

(iii) an alcohol of formula (X₁),—OH or an

alkoxy moiety of formula —(X₁)ₙ—O—X₂, where X₁ and X₂ are independently selected from the group consisting of hydrogen, saturated or unsaturated alkyl, and homocyclic or heterocyclic ring moieties, where the ring is optionally substituted with one or more substituents independently selected from the group consisting of alkyl, alkoxy, halogen, trihalomethyl, carboxylate, nitro, and ester and where n is 0 or 1;

(iv) a homocyclic or heterocyclic ring moiety optionally substituted with one, two, or three substituents independently selected from the group consisting of alkyl, alkoxy, halogen, trihalomethyl, carboxylate, nitro, and ester moieties;

(v) halogen or trihalomethyl;

(vi) a thioether of formula —SX₃, where X₃ is selected from the group consisting of hydrogen, saturated or unsaturated alkyl, and homocyclic or heterocyclic ring moieties, where the ring is optionally substituted with one or more substituents independently selected from the group consisting of alkyl, alkoxy, halogen, trihalomethyl, carboxylate, nitro, and ester;

(vii) a sulfoxide of formula —S(O)X₄, where X₄ is selected from the group consisting of alkyl and aryl;

(viii) a sulfone of formula —SO₂—X₅, where X₅ is selected from the group consisting of saturated or unsaturated alkyl and homocyclic or heterocyclic ring moieties;

(ix) —SO₂X₆, where X₆ is selected from the group consisting of hydrogen, saturated or unsaturated alkyl, and homocyclic or heterocyclic ring moieties, where the ring is optionally substituted with one or more substituents independently selected from the group consisting of alkyl, alkoxy, halogen, trihalomethyl, carboxylate, nitro, and ester;

(x) —SO₂NX₇X, where X₇ and X₈ are selected from the group consisting of hydrogen, alkyl, and homocyclic or heterocyclic ring moieties;

(xi) nitro;

(xii) NX₉X₁₀, where X₉ and X₁₀ are independently selected from the group consisting of hydrogen, saturated or unsaturated alkyl, and homocyclic or heterocyclic ring moieties;

(xiii) cyano;

(xiv) a ketone of formula —CO—X₁₁, where X₁₁ is selected from the group consisting of hydrogen, alkyl, and homocyclic or heterocyclic ring moieties;

(xv) a carboxylic acid of formula —(X₁₂)ₙ—COOH or ester of formula —(X₁₃)ₙ —COO—X₁₄, where X₁₂, X₁₃, and X₁₄ and are independently selected from the group consisting of alkyl and homocyclic or heterocyclic ring moieties and where n is 0 or 1;

(xvi) an amide of formula —NHCOX₁₅, where X₁₅ is selected from the group consisting of alkyl, hydroxyl, and homocyclic or heterocyclic ring moieties, where the ring is optionally substituted with one or more substituents independently selected from the group consisting of alkyl, alkoxy, halogen, trihalomethyl, carboxylate, nitro, and ester; and

(xvii) an aldehyde of formula —CO—H; and

d) A is a homocyclic or heterocyclic ring moiety optionally substituted with one, two, or three substituents independently selected from the group consisting of alkyl, alkoxy, halogen, trihalomethyl, carboxylate, nitro, and ester moieties.

The term “indolinone” is used as that term is commonly understood in the art and includes a large subclass of substituted or unsubstituted compounds that are capable of being synthesized from an aldehyde moiety and an oxindole moiety.

The term “saturated alkyl” refers to an alkyl moiety that does not contain any alkene or alkyne moieties. The alkyl moiety may be branched or non-branched.

The term “unsaturated alkyl” refers to an alkyl moiety that contains at least one alkene or alkyne moiety. The alkyl moiety may be branched or non-branched.

The term “alcohol” refers to a chemical substituent of formula —ROH, where R is selected from the group consisting of hydrogen, saturated or unsaturated alkyl, and homocyclic or heterocyclic ring moieties, where the ring moiety is optionally substituted with one or more substituents independently selected from the group consisting of alkyl, halogen, trihalomethyl, carboxylate, nitro, and ester moieties.

The term “alkoxy moiety” refers to a chemical substituent of formula —OR, where R is hydrogen or a saturated or unsaturated alkyl moiety.

The term “halogen” refers to an atom selected from the group consisting of fluorine, chlorine, bromine, and iodine.

The term “sulfone” refers to a chemical moiety with formula —SO₂—R, where R is selected from the group consisting of saturated or unsaturated alkyl and homocyclic or heterocyclic ring moieties.

The term “nitro” refers to a chemical moiety with formula —NO₂.

The term “cyano” refers to a chemical moiety with formula —CN.

The term “ketone” refers to a chemical moiety with formula —(R)n—CO—R′, where R and R′ are selected from the group consisting of saturated or unsaturated alkyl and homocyclic or heterocyclic ring moieties and where n is 0 or 1.

The term “carboxylic acid” refers to a chemical moiety with formula —(R)n—COOH, where R is selected from the group consisting of saturated or unsaturated alkyl and homocyclic or heterocyclic ring moieties, and where n is 0 or 1.

The term “amide” refers to a chemical substituent of formula —NHCOR, where R is selected from the group consisting of hydrogen, alkyl, hydroxyl, and homocyclic or heterocyclic ring moieties, where the ring is optionally substituted with one or more substituents independently selected from the group consisting of alkyl, halogen, trihalomethyl, carboxylate, nitro, or ester.

The term “ester” refers to a chemical moiety with formula —(R)n—COOR′, where R and R′ are independently selected from the group consisting of saturated or unsaturated alkyl and homocyclic or heterocyclic ring moieties and where n is 0 or 1.

The term “aldehyde” refers to a chemical moiety with formula —(R)n—CHO, where R is selected from the group consisting of saturated or unsaturated alkyl and homocyclic or heterocyclic ring moieties and where n is 0 or 1.

In preferred embodiments, the indolinone compound is selected from the group consisting of

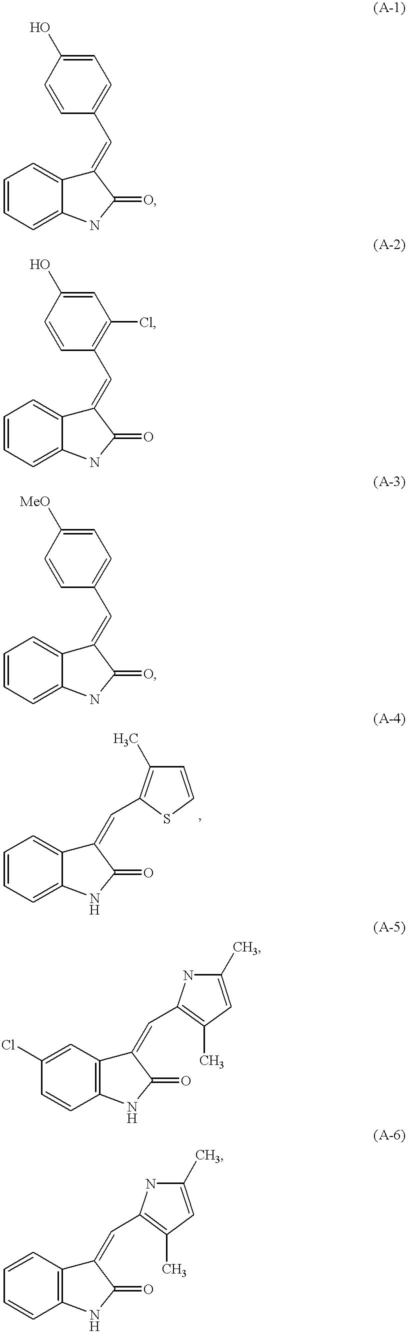

In other preferred embodiments, the physico-chemical property is selected from the group consisting of solubility, partition coefficient, pKₐ, pH-solubility, and pH-stability.

The term “solubility” refers to the ability of a compound to dissolve in a given solvent.

The term “partition coefficient” refers to ratio of the concentrations of a compound in different solvents, when that compound was dissolved in a solvent system comprising of two or more immiscible solvents.

The term “pH-solubility” refers to the solubility of a compound in solutions of varying acidity. Some compounds become more soluble in a solution as the solution becomes more acidic while others become less soluble. The solubility of other compounds, yet, remains unchanged as the acidity of the solution is varied.

The term “pH-stability” refers to the stability of a compound in solutions of varying acidity. Some compounds in solution chemically decompose as the solution becomes more acidic while others decompose as the solution becomes more basic. Still other compounds retain their chemical integrity regardless of the acidity of the solution.

The invention also preferably allows for the determination of a compound most suitable as a drug. The result of a preformulation study of a compound, such as the compound's pKₐ, solubility, etc., is one of the criteria used in choosing a suitable potential drug candidate.

The summary of the invention described above is non-limiting and other features and advantages of the invention will be apparent from the following description of the preferred embodiments, and from the claims.

### BRIEF DESCRIPTION OF THE FIGURES

FIGS. 1a and 1b show two series of solutions with a different known compound dissolved in each series. In FIG. 1a, compound A is dissolved in series a, under 3 different conditions. In FIG. 1b, compound B is dissolved in series b, under 3 different conditions.

FIG. 1c shows that a single solution from one series is mixed with the corresponding solution from each of the other series.

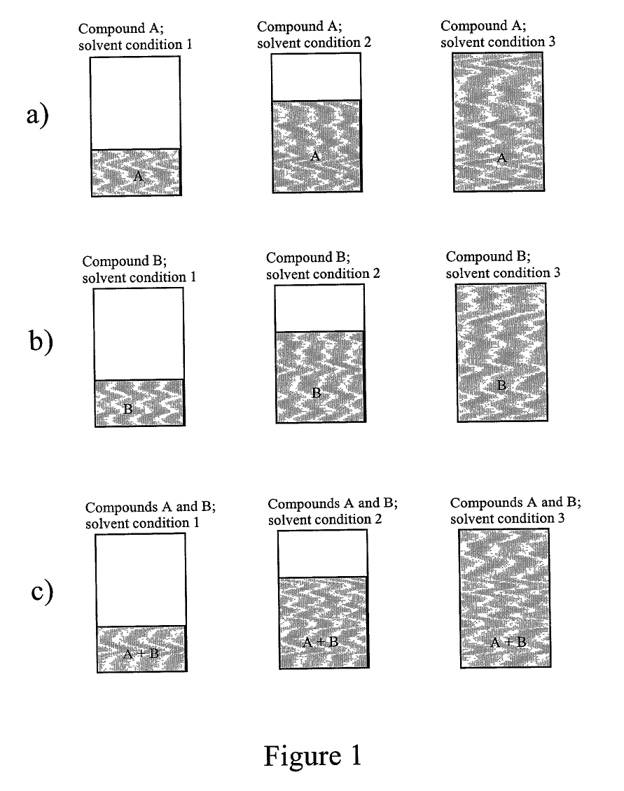

### DETAILED DESCRIPTION OF THE INVENTION

The present invention is directed in part towards a method for high-throughput pharmaceutical preformulations. Traditionally, preformulation analyses on potential drugs have involved the study of a single compound under the appropriate conditions. This process is very time consuming if one is testing a large set of potential drugs. An advantage of the methods of the present invention is that it allows for the simultaneous analysis of a large number of compounds. The methods of the present invention can be practiced on any combination of compounds. The following detailed description is not meant to limit the practice of the invention to compounds used in connection with a specific disease or biochemical process.

I. High Pressure Liquid Chromatographic Analysis

The high pressure liquid chromatograph (HPLC) is an instrument well known to those skilled in the art. This invention makes use of HPLC in order to determine the concentrations of the compounds under analysis. Those skilled in the art know how to vary the conditions of HPLC, such as flow rate, solvent ratio, column composition, injection volume, and detection frequency, in order to optimize the results based on the specific set of compounds under study. To exemplify these conditions, a set of HPLC conditions are presented herein in the Examples.

II. Biological Activity of Indolinone Compounds

Indolinone compounds of the invention can be tested for their ability to activate or inhibit protein kinases, such as the FLK protein kinase, in biological assays. The indolinone compounds listed herein have been shown to inhibit vascular endothelial growth factor (VEGF) induced endothelial cell growth in vitro studies and could be potential angiogenesis inhibitors.

### EXAMPLES

The examples below are not limiting and are merely representative of various aspects and features of the present invention. The examples demonstrate methods of practicing the invention.

### Example 1

### Simultaneous Analysis of Potential Drug Candidates Using HPLC

A reverse phase HPLC method that simultaneously analyzes potential drug candidates is developed to allow for high-throughput preformulation studies to accelerate drug discovery and development. A Hewlett Packard 1090 HPLC equipped with a photodiode array detector, a ternary solvent delivery system, and column heater was used. The detector was capable of simultaneous detection at eight (8) different wavelengths in the 190-650 nm range.

Sample Preparations

Aliquots of volumetrically made solutions of the different compounds were mixed in volumetric flasks. The solution mixture was quantitatively diluted to volume with acetonitrile. The dilution factor was factored into the calculations for estimation of the final concentrations.

Calibration Standard Preparations

A standard solution of the individual compounds was prepared volumetrically in acetonitrile. Serial dilutions of the standard mixture was made. The concentrations investigated, ranged from 0.1 to 50.0 μg/mL.

HPLC Method

Method: Reverse phase isocratic HPLC method

Column: C₁₈, 250×4.6 mm, Hypersil column at 40° C.

Mobile Phase: 55:45, 0.1% trifluoroacetic acid: acetonitrile

Flow rate: 0.8 mL/min

Injection Volume: 10 μL

Detection: 425 nm, 350 nm, 254 nm (other wavelengths in the UV-Visible region)

The above conditions were used in order to obtain the data in Table 1. However, those skilled in the art know that it is possible to use other conditions. For example, columns with different types of stationary phases like the C₁₈, C₈, Cyano, and Phenyl can be used. The column length and diameter can be varied from 75 to 250 mm and 4.6 to 1.0 mm, respectively. The particle size of the stationary phase can be varied from 3.5 to 10μ. The column can be used at temperatures ranging from room temperature to 55° C.

Furthermore, different types of aqueous phases not absorbing in the UV-Visible range (or the detection range desired) can be used. Examples include phosphate buffers, acetate buffers, acidic or basic aqueous phases (pH 2.5-8). Ion-pairing reagents like triethylamine can be added to the aqueous phase. Different organic mobile phases not absorbing in the UV-Visible range (or the detection range desired) like methanol, acetonitrile, or tetrahydrofuran can be used. The proportion of aqueous to organic phases or the flow rates can be varied isocratically or in a gradient to optimize the chromatography.

Additionally, different types of detectors can be used, for example a mass spectrometer can be used with volatile mobile phases or a refractive index detector can be used for compounds that do not absorb well in the UV range. The most popular detectors are the UV-Visible detector for compounds that absorb in the UV range. Multi-wavelength detectors can detect compounds absorbing in different ranges.

Those skilled in the art know that these methods can be customized for a group of compounds by varying the different chromatographic parameters discussed above.

Table 1 summarizes the chromatographic parameters of the drug candidates studied. Some of the candidates exhibited cis-trans isomerism. All of the compounds, and their respective cis-trans isomers are separated by the reverse phase HPLC method. The behavior of the compounds was linear in the 1-50 μg/mL, at the three detection wavelengths 425 nm, 350 nm and 254 nm. The limit of quantification (LOQ) for the compounds A-1, A-2, A-3, A-4, A-6, and A-7 at 350 nm was 0.5 μg/mL. The limit of quantification for A-8 at 254 nm was 0.5 μg/mL. The limit of detection (LOD) for the compounds A-1, A-2, A-3, A-4, and A-7 at 350 nm was 0.1 μg/mL. LOD for A-6 and A-8 was 0.1 μg/mL at 425 nm and 254 nm respectively.

The reverse phase HPLC method, therefore, allows for sensitive and simultaneous quantification of potential drug candidates. It serves as an analytical tool for high-throughput preformulation studies such as solubility studies, pKₐ determinations, partition co-efficient determinations and pH-solubility /stability studies. The method can be adapted to LC-MS analysis for multiple applications.

TABLE 1

| | | | | | | LOQ (μg/mL) | | | | | |
|---|---|---|---|---|---|---|---|---|---|---|---|
| | RT (mins) | | *r² | | | 425 | 350 | 254 | LOD (μg/mL) | | |
| Compd. | Ma | Mi | 425 nm | 350 nm | 254 nm | nm | nm | nm | 425 nm | 350 nm | 254 nm |
| A-1 | 4.2 | 6.0 | 0.999 | 0.999 | 0.999 | 0.5-1.0 | 0.5 | 0.5 | 0.1-0.5 | 0.1 | 0.1 |
| A-2 | 5.4 | 9.7 | 0.999 | 0.999 | 0.999 | 0.5-1.0 | 0.5 | 0.5 | 0.1-0.5 | 0.1-0.5 | 0.1 |
| A-3 | 9.9 | 13.2 | 0.992 | 0.999 | 0.999 | 0.5-1.0 | 0.5 | 0.5 | 0.1-0.5 | 0.1 | 0.1 |
| A-4 | 11.3 | 12.6 | 0.999 | 0.999 | 0.999 | 0.5-1.0 | 0.5 | 0.5 | 0.1-0.5 | 0.1-0.5 | 0.1 |
| A-5 | 44.2 | — | 0.999 | 0.999 | 0.999 | 0.5-1.0 | 1.0-5.0 | 1.0-5.0 | 0.1-0.5 | 0.5-1.0 | 0.5-1.0 |
| A-6 | 23.6 | — | 0.999 | 0.999 | 0.998 | 0.5 | 0.5 | 0.5-1.0 | 0.1 | 0.1 | 0.1 |
| A-7 | 13.7 | 13.1 | 0.999 | 0.999 | 0.999 | 0.5 | 0.5-1.0 | 0.5-1.0 | 0.1-0.5 | 0.1-0.5 | 0.1 |
| | | | (5-50) | | | | | | | | |
| A-8 | 20.5 | 25.1 | 0.992 | 0.999 | 0.999 | 0.5-1.0 | 0.5 | 0.5 | 0.1-0.5 | 0.1-0.5 | 0.1 |

*Linearity range 1-50 μg/mL, LOQ: Limit of quantification, LOD: Limit of detection, Ma: Major peak, Mi: Minor Peak, The earlier eluting peak is the trans isomer, RT: Retention time

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments and are exemplary and are not intended as limitations on the scope of the invention. Changes therein and other uses will occur to those skilled in the art which are encompassed within the spirit of the invention and are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

What is claimed is:

1. A method of simultaneous evaluation of a plurality of potential drug candidates, comprising the steps of:
(a) forming multiple series of solutions, each series of solutions containing a different known compound in a solvent or in a mixture of solvents under varying solution conditions;
(b) mixing a single solution from one series of solutions with the corresponding solution from each of the other series of solutions, thereby forming a mixture of solutions, wherein said corresponding solutions have substantially similar solution conditions;
(c) separately injecting an aliquot of each of said mixture of solutions into a high pressure liquid chromatograph;
(d) determining the concentration of each of said different compounds in each of said mixture of solutions by analysis of results from said injection; and
(e) correlating the concentration of each of said different compounds in each of said mixture of solutions to a physico-chemical property of said compound.

2. The method of claim 1, wherein said solution conditions are selected from the group consisting of pH, polarity, dielectric constant, and temperature.

3. The method of claim 1, wherein said compound is an indolinone compound.

4. The method of claim 3, wherein said indolinone compound is a compound of formula I 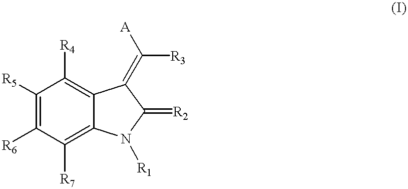
wherein
a) R₁ and R₃ are independently selected from the group consisting of hydrogen and saturated or unsaturated alkyl;
b) R₂ is selected from the group consisting of oxygen and sulfur;
c) R₄, R₅, R₆, and R₇ are independently selected from the group consisting of
(i) hydrogen;
(ii) saturated or unsaturated alkyl;
(iii) an alcohol of formula (X₁)ₙ-OH or an alkoxy moiety of formula —(X₁)ₙ—O—X₂, where X₁ and X₂ are independently selected from the group consisting of hydrogen, saturated or unsaturated alkyl, and homocyclic or heterocyclic ring moieties, wherein said ring is optionally substituted with one or more substituents independently selected from the group consisting of alkyl, alkoxy, halogen, trihalomethyl, carboxylate, nitro, and ester and where n is 0 or 1;
(iv) a homocyclic or heterocyclic ring moiety optionally substituted with one, two, or three substituents independently selected from the group consisting of alkyl, alkoxy, halogen, trihalomethyl, carboxylate, nitro, and ester moieties;
(v) halogen or trihalomethyl;
(vi) a thioether of formula —SX₃, where X₃ is selected from the group consisting of hydrogen, saturated or unsaturated alkyl, and homocyclic or heterocyclic ring moieties, wherein said ring is optionally substituted with one or more substituents independently selected from the group consisting of alkyl, alkoxy, halogen, trihalomethyl, carboxylate, nitro, and ester;
(vii) a sulfoxide of formula —S(O)X₄, where X₄ is selected from the group consisting of alkyl and aryl;
(viii) a sulfone of formula —SO₂—X₅, where X₅ is selected from the group consisting of saturated or unsaturated alkyl and homocyclic or heterocyclic ring moieties;
(ix) —SO₂X₆, where X₆ is selected from the group consisting of hydrogen, saturated or unsaturated alkyl, and homocyclic or heterocyclic ring moieties, wherein said ring is optionally substituted with one or more substituents independently selected from the group consisting of alkyl, alkoxy, halogen, trihalomethyl, carboxylate, nitro, and ester;
(x) —SO₂NX₇X₈, where X₇ and X₈ are selected from the group consisting of hydrogen, alkyl, and homocyclic or heterocyclic ring moieties;
(xi) nitro;
(xii) NX₉X₁₀, where X₉ and X₁₀ are independently selected from the group consisting of hydrogen, saturated or unsaturated alkyl, and homocyclic or heterocyclic ring moieties;
(xiii) cyano;
(xiv) a ketone of formula —CO—X₁₁, where X₁₁ is selected from the group consisting of hydrogen, alkyl, and homocyclic or heterocyclic ring moieties;
(xv) a carboxylic acid of formula —(X₁₂)—COOH or ester of formula —(X₁₃)ₙ—COO—X₁₄, where X₁₂, X₁₃, and X₁₄ and are independently selected from the group consisting of alkyl and homocyclic or heterocyclic ring moieties and where n is 0 or 1;
(xvi) an amide of formula —NHCOX₁₅, where X₁₅ is selected from the group consisting of alkyl, hydroxyl, and homocyclic or heterocyclic ring moieties, wherein said ring is optionally substituted with one or more substituents independently selected from the group consisting of alkyl, alkoxy, halogen, trihalomethyl, carboxylate, nitro, and ester; and
(xvii) an aldehyde of formula —CO—H; and
d) A is a homocyclic or heterocyclic ring moiety optionally substituted with one, two, or three substituents independently selected from the group consisting of alkyl, alkoxy, halogen, trihalomethyl, carboxylate, nitro, and ester moieties.

5. The method of claim 4, wherein said indolinone compound is selected from the group consisting of

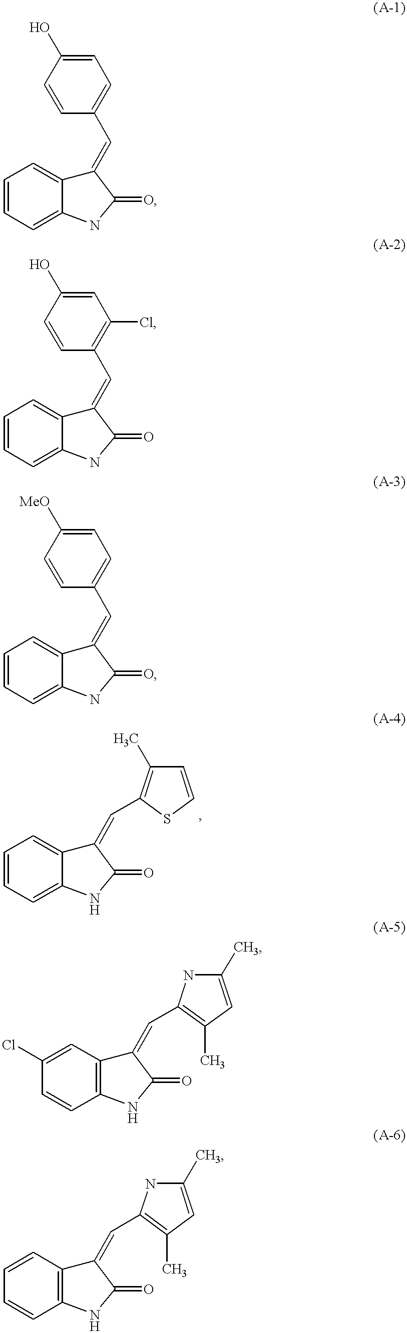

6. The method of claim 1, wherein said physico-chemical property is selected from the group consisting of solubility, partition coefficient, pKₐ, pH-solubility, and pH-stability.

7. The method of claim 1, comprising determining the compound most suitable as a drug.

8. A drug for treating a disease, wherein said drug was chosen based on the simultaneous evaluation of the method of claim 7.
